(19) United States
(12) Patent Application Publication
(10) Pub. No.: US 2022/0061763 A1
(43) Pub. Date: Mar. 3, 2022

(54) OPTICAL FUNCTIONAL GUIDEWIRE, DETECTION SYSTEM, AND DETECTION METHOD

(71) Applicant: Hua Shang, Nanjing (CN)

(72) Inventor: Hua Shang, Nanjing (CN)

(73) Assignee: Hua Shang, Nanjing (CN)

(21) Appl. No.: 17/395,134

(22) Filed: Aug. 5, 2021

Related U.S. Application Data
(63) Continuation of application No. PCT/CN2020/134600, filed on Dec. 8, 2020.

(30) Foreign Application Priority Data
Aug. 31, 2020 (CN) 202010894792.7

(51) Int. Cl. A61B 5/00; A61M 25/09

(52) U.S. Cl. CPC A61B 5/6851; A61M 25/09016; A61M 2205/3306; A61M 2025/09133; A61B 5/0084

(57) ABSTRACT

The disclosure provides an optical functional guidewire, a detection system and a detection method. The optical functional guidewire includes an optical fiber and a sleeve surrounding the optical fiber. The optical fiber includes a functional section capable of emitting and collecting laser light. The functional section is provided with at least one grating assembly. The sleeve includes a shaping section capable of bending and a supporting section capable of supporting advancement of the functional section. The shaping section is connected to the functional section and is located at an end close to the functional section. The supporting section is located at an end away from the functional section. The optical functional guidewire provided in this disclosure has good bending performance and operability, and thus can be easily manipulated, readily enters a body cavity with a larger opening angle, and achieves self-guidance and flexible detection of the optical functional guidewire in the body cavity, thereby improving the effect of minimally invasive interventional treatment.

### TECHNICAL FIELD

The disclosure relates to the technical field of medical devices, in particular to an optical functional guidewire, a detection system and a detection method.

### BACKGROUND

Minimally invasive interventional therapy is a medical technology that uses specific devices such as a puncture needle, guidewire or catheter under imaging guidance to accurately reach lesion sites for diagnosis and treatment without open surgery to human body. Minimally invasive interventional therapy is more and more favored by patients for its characteristic such as definite curative effect, fast recovery, strong targeting, low recurrence, no side effects, small trauma, safety and reliability, low cost and the like.

The guidewire is frequently used in clinical practice. For example, the guidewire may be used for assisting the installation of heart stents, the ablation of thrombus, and the treatment of tumor embolization. As for the interventional surgery, the safety of the guidewire comes first. Therefore, the guidewire must have many characteristics, such as flexible head portion, good compliance, no damage, high plasticity, and providing low to moderate support.

At present, the medical guidewire sold on the market is usually composed of a wire of stainless steel in a core. Such a wire of stainless steel has multiple sections of different diameters and is formed to the medical guidewire by winding at top end of the wire. However, these solutions will cause the guidewire to have a larger diameter, making it difficult to enter blood vessel having a small diameter.

Meanwhile, the guidewire is usually provided with a head portion that can be actively bent, in order to achieve good maneuverability in cavity of the human body. Therefore, a shape of the head portion can be changed according to the direction of the body cavity, and thus can be easy to enter smaller branch body cavity. Currently, the guidewire is mainly driven by multiple tendons, magnetic field or memory metal to guide it to follow the established route in the body cavity, which has great limitation to operations. Therefore, how to improve the operation performance, driving performance and detection performance of the guidewire has become an urgent problem to be solved.

### SUMMARY

In view of the above, embodiments of the disclosure provide an optical functional guidewire, a detection system, and a detection method, so as to solve technical defects in the related art.

The disclosure provides an optical functional guidewire. The optical functional guidewire includes an optical fiber and a sleeve surrounding the optical fiber. The optical fiber includes a functional section capable of emitting and collecting laser light. The functional section is provided with at least one grating assembly. The sleeve includes a shaping section capable of bending and a supporting section capable of supporting an advancement of the functional section. The shaping section is connected to the functional section and is located at an end close to the functional section. The supporting section is located at an end away from the functional section. The optical functional guidewire is further provided with an asymmetric structure capable of directional bending of the optical functional guidewire.

Optionally, the functional section is provided with multiple grating assemblies. The grating assemblies are sleeved on the functional section of the optical functional guidewire at intervals, and are arranged longitudinally along the optical fiber.

Optionally, the optical fiber includes a core layer located at an axis, and a cladding layer surrounding the core. The grating assemblies are sleeved outside the cladding layer, and each of grating assemblies is in a shape of hollow prism.

Optionally, the grating assembly include a plurality of gratings with different periods, and each grating constitutes a side surface of the grating assembly.

Optionally, a diameter of the supporting section is larger than a diameter of the shaping section.

Optionally, the sleeve further includes a transition section and a pushing section. The transition section is located between the shaping section and the supporting section, and a diameter of the transition section gradually increases in a direction from the shaping section to the supporting section. An end of the pushing section is connected to the supporting section, and other end of the pushing section is connected to a driving mechanism.

Optionally, the functional section of the optical fiber is connected to the shaping section of the sleeve via a spiral tube. A developing ring is provided between the spiral tube and the optical fiber.

Optionally, the asymmetric structure is an asymmetric tube wall structure of the sleeve.

Optionally, the asymmetric tube wall structure is an asymmetric slit opened on the shaping section of the sleeve; the asymmetric slit is a spiral slit or a rectangular slit. In the case of the asymmetric slit being the spiral slit, the asymmetric slit has different widths on two sides of the sleeve, while in the case of the asymmetric slit being the rectangular slit, the asymmetric slit has different depths on two sides of the sleeve.

Optionally, the asymmetric tube wall structure is formed by a wall thickness on one side of the sleeve being smaller than a wall thickness of other side of the sleeve.

Optionally, the asymmetric tube wall structure is formed by a shape of the sleeve having a convex side and a planar side, or having a convex side and a concave side. The convex side has an arched structure.

Optionally, the functional section, at an end away from the shaping section, is provided with a hemispherical optical component capable of blocking laser scattering. A polymer coating is provided outside the optical functional guidewire. The polymer coating can be a hydrophilic coating or a hydrophobic coating.

Optionally, the sleeve is a hypotube, has an outer diameter of 0.6-0.8 mm, and has an inner diameter of 0.3-0.5 mm.

The disclosure also provides a detection system including:

the optical functional guidewire as mentioned above;

a control center arranged for sending control signals to an attitude controller, a multi-wavelength pulsed laser, a waveform collector, and a treatment laser to control a start-up, operation or shutdown of the attitude controller, the multi-wavelength pulsed laser, the waveform collector, and the treatment laser;

the attitude controller arranged for receiving the signals sent by the control center and distance information, and driving the optical functional guidewire to entry or exit a body cavity or move in the body cavity;

the multi-wavelength pulsed laser arranged for receiving the signals sent by the control center, and sending out pulsed laser light which is transmitted to the optical functional guidewire, and is scattered into a body cavity through the grating assembly (8) of the optical functional guidewire;

the waveform collector arranged for receiving the signals sent by the control center, analyzing a delayed waveform of scattered laser in the body cavity through the grating assembly of the optical functional guidewire, to obtain position information about a wall of the body cavity and the optical functional guidewire, and feedback the position information to the control center.

Optionally, the multi-wavelength pulsed laser and the waveform collector are coupled to the optical fiber via a fiber splitting coupler.

The disclosure also provides a detection method used in the detection system mentioned above. The method includes the following operations.

The control center receives control instructions, and sends control signals to the attitude controller and the multi-wavelength pulsed laser based on the control instructions.

The attitude controller receives the control signals sent by the control center, and drives the optical functional guidewire into the body cavity based on the control signals.

The pulsed detector receives the control signals sent by the control center, emits pulsed laser light, and scatters the pulsed laser light into the body cavity via the optical functional guidewire and the grating assembly.

The optical functional guidewire receives a reflected pulsed laser light and sends the reflected pulsed laser light to the waveform collector. Based on the reflected pulsed laser light, the waveform collector determines a position of the optical functional guidewire in the body cavity.

The attitude controller controls a subsequent movement of the optical functional guidewire based on the position of the optical functional guidewire in the body cavity, until the optical functional guidewire reaches target site and exits the body cavity after completing detection.

The optical functional guidewire provided in this disclosure includes at least one optical fiber and a sleeve surrounding the optical fiber. The optical fiber has a functional section of emitting and collecting laser light. The functional section is provided with at least one grating assembly. The grating assembly has a function of emitting and collecting detection laser light, and can be used for determining the distance between the wall of the cavity and the optical fiber by emitting and collecting lasers having different specific wavelengths and analyzing the time waveform obtained thereby, so as to guide the optical functional guidewire to change the shape and attitude over time, thereby realizing the intelligent-guidance and detection in the body cavity. The sleeve includes the functional section, the guiding section and the supporting section which are connected in sequence. In addition, in order to improve the bending performance and operability of the optical functional guidewire, the asymmetric structure is provided on the sleeve itself or the surround of the sleeve along the optical fiber, so that the optical functional guidewire can be easily manipulated and can readily enter the body cavity with a larger opening angle, and precise detection and treatment can be carried out in the body cavity through laser transmission, thereby improving the effect of minimally invasive interventional treatment.

The detection system provided by this disclosure includes the optical functional guidewire, the control center, the attitude controller, the multi-wavelength pulsed laser, and the waveform collector. Among them, the control center can send control signals to other components to coordinate and control the cooperation among these components. The attitude controller can control the optical functional guidewire to enter or exit the body cavity or move in the body cavity, which improves the flexibility of the optical functional guidewire during use. Through the cooperation of the multi-wavelength pulsed laser, the waveform collector and the optical functional guidewire, the relative position of the optical functional guidewire and the wall of the body cavity can be determined by the delay of the laser light, and then the subsequent attitude and moving direction of the optical functional guidewire can be accurately determined. The detection system provided by this disclosure innovatively uses light to guide the travel of the guidewire, and has high detection efficiency and good detection effect.

The detection method provided in this disclosure realizes the intelligent and automatic guidance of the optical functional guidewire in the body cavity through the cooperation of the control center, the attitude controller, the pulsed detector, the optical functional guidewire and the waveform collector. The method of the disclosure is easy and convenient in operation, and greatly improves the detection efficiency and detection effect of the optical functional guidewire. In addition, through the cooperation of the control center, the optical functional guidewire and the treatment laser, the laser irradiation treatment can be performed to the lesion sites of the patient with high treatment efficiency and good effect, thereby improving flexibility in use and application range of the optical functional guidewire.

### BRIEF DESCRIPTION OF THE DRAWINGS

FIG. 1 is an overall schematic diagram of the optical functional guidewire according to an example of the disclosure;

FIG. 2 is a local schematic diagram of the optical functional guidewire according to an example of the disclosure;

FIG. 3 is a schematic diagram of the structure of the grating assembly according to an example of the disclosure;

FIG. 4 is a schematic diagram of the optical functional guidewire according to an example of the disclosure in use;

FIG. 5 is a schematic diagram for detecting the distance by the pulse according to an example of the disclosure;

FIG. 6 is a local schematic diagram of the optical functional guidewire according to an example of the disclosure;

FIG. 7 is another overall schematic diagram of the optical functional guidewire according to an example of the disclosure;

FIG. 8 is another overall schematic diagram of the optical functional guidewire according to an example of the disclosure;

FIG. 9 is another overall schematic diagram of the optical functional guidewire according to an example of the disclosure;

FIG. 10 is another overall schematic diagram of the optical functional guidewire according to an example of the disclosure;

FIG. 11 is another overall schematic diagram of the optical functional guidewire according to an example of the disclosure;

FIG. 12 is a working principle diagram of the detection system according to an example of the disclosure;

FIG. 13 is a delayed waveform of superimposed laser pulse according to an example of the disclosure.

### LIST OF REFERENCE SYMBOLS

1, optical fiber; 2, sleeve; 3, functional section; 4, shaping section; 5, supporting section; 6, transition section; 7, pushing section; 8, grating assembly; 9, core layer; 10, cladding layer; 11, spiral tube; 12, developing ring; 13, polymer coating; 14, hemispherical optical component; 15, asymmetric slit; 16, convex side; 17, planar side.

### DETAILED DESCRIPTION

The embodiments of the disclosure are described below with reference to the drawings.

In this disclosure, unless otherwise specified, the scientific and technical terms used herein have the meanings commonly understood by those skilled in the art. In addition, the reagents, materials and operation steps used in this disclosure belong to common reagents, materials and routine procedures widely used in the corresponding field. Furthermore, in order to better understand the disclosure, definitions and explanations of related terms are provided below.

In this disclosure, hypotube refers to a long metal tube with micro-engineering characteristics throughout the tube. It is an important component of a catheter for minimally invasive treatment and is used to dredge clogged arteries in conjunction with a balloon and a stent. The balloon of the catheter is attached to a distal end of the hypotube. The hypotube enters the human body and pushes the balloon toward the clog portion in the artery along the long tortuous blood vessel. During this operation, it is required to avoid the kinking of the hypotube, and enable it to move smoothly in the human body (propulsion, tracking and rotation).

### Example 1

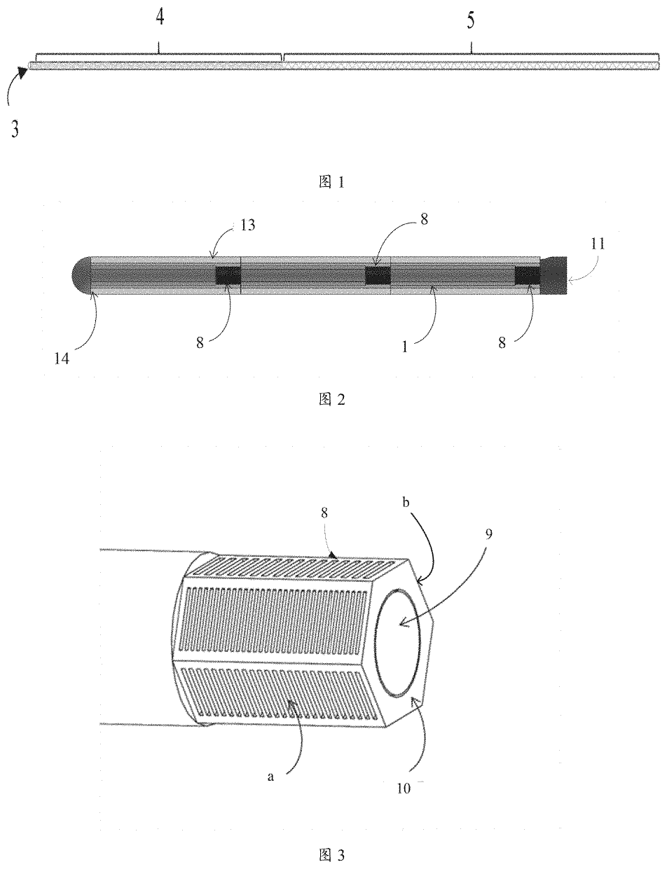

The present example provides an optical functional guidewire. As shown in FIG. 1, the optical functional guidewire includes an optical fiber 1 and a sleeve 2 surrounding the optical fiber 1. The optical fiber 1 includes a functional section 3 capable of emitting and collecting laser light. The functional section 3 is provided with at least one grating assembly 8. The sleeve 2 includes a shaping section 4 capable of bending and a supporting section 5 capable of supporting an advancement of the functional section 3. The shaping section 4 is connected to the functional section 3 and is located at an end close to the functional section 3. The supporting section 5 is located at an end away from the functional section 3. The optical functional guidewire is further provided with an asymmetric structure capable of directional bending of the optical functional guidewire.

In this example, the optical fiber 1 may be an artificial fiber for transmitting light, and is located at an axis of the optical functional guidewire. The functional section 3 of the optical fiber 1 can emit and collect laser light through the grating assembly 8 provided thereon, and thus can determine the position of the optical functional guidewire in the body cavity according to the time waveform produced by laser light.

As shown in FIG. 2, there may be one or more the grating assembly 8, such as 2, 3, 4, 5, etc., preferably 3. In the case that there are multiple grating assemblies 8, the grating assemblies 8 are sleeved outside the functional section 3 of the optical functional guidewire at intervals, and are arranged longitudinally along the optical fiber. The interval between two adjacent grating assemblies 8 can be determined, depending on specific circumstances, which is not limited in this disclosure.

More specifically, the optical fiber 1 includes a core layer 9 located at an axis, and a cladding layer 10 surrounding the core layer 9. The adjacent grating assemblies 8 are sleeved outside the cladding layer 10, and each of grating assemblies 8 is in a shape of hollow prism. The cladding layer 10 is made of a transparent polymer, so that laser light of the optical fiber 1 can pass through the cladding layer 10 and scatter into the body cavity via the grating assemblies 8. The grating assemblies 8 may be in the shape of a hollow quadrangular prism, a hollow hexagonal prism, a hollow octagonal prism, a hollow ten prism, etc., and preferably a hollow hexagonal prism.

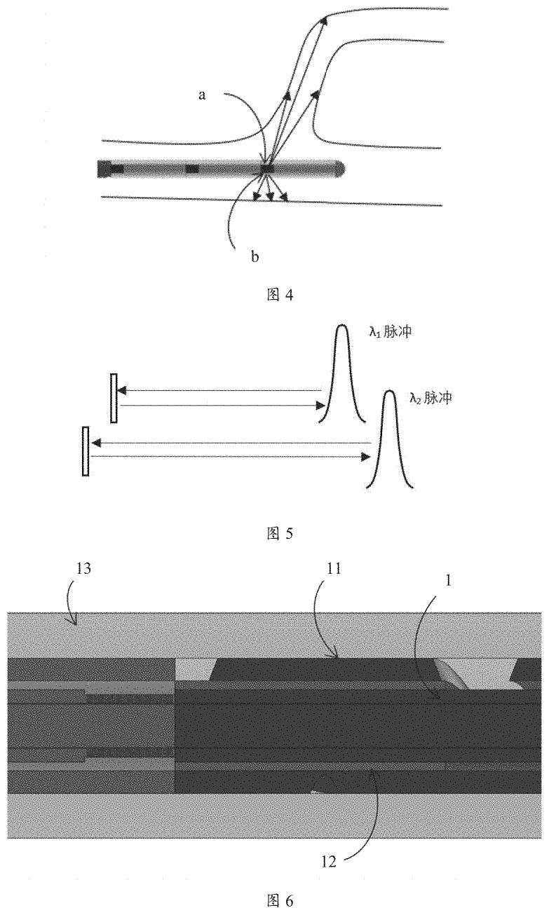

The structure of the grating assembly 8 is shown in FIG. 3. The grating assembly 8 include a plurality of gratings. The grating is an optical device that is fixed on the optical fiber 1 for emitting and collecting laser light, and is composed of a large number of parallel slits having equal width and equal spacing.

Each of the grating assemblies 8 includes a plurality of gratings with different periods, and each grating constitutes a side surface of the prismatic cladding layer 10. When pulsed lasers with multiple wavelengths are transmitted into the optical fiber, the wavelengths of the pulses coupled from different gratings are different. The number of gratings of the grating assembly 8 is the same as the number of side surfaces of the prism. For example, when the grating assembly 8 is in the shape of a hollow hexagonal prism, it is composed of 6 gratings with different periods.

Referring to FIG. 3, a and b represent two gratings in opposite directions. In the practices, the laser light emitted by grating a is scattered by the body cavity wall and then coupled into the optical fiber via the grating a, while the laser light emitted by grating b is scattered by the cavity wall, and then coupled into the optical fiber via grating b. In the case of a branch cavity at grating a, as shown in FIG. 4, the distance between the grating a and the cavity wall is greater than the distance between the grating b and the cavity wall, and thus the time for collecting the scattered pulses by grating a is lagging behind that by grating b. As shown in FIG. 5, λ1 represents the wavelength of the light emitted by grating a, and λ2 represents the wavelength of the light emitted by grating b. By analyzing the waveform of the scattered echo, the branch morphology of the cavity can be obtained, thereby guiding the shaping section 4 to bend to entry into the branch cavity. By analyzing the waveform of the grating echo in different directions, the situation of the branch cavity where each grating is located can be determined, thereby providing more detailed judgment data for the more complicated shape of passage of the cavity, so as to improve the efficiency of movement of the guidewire.

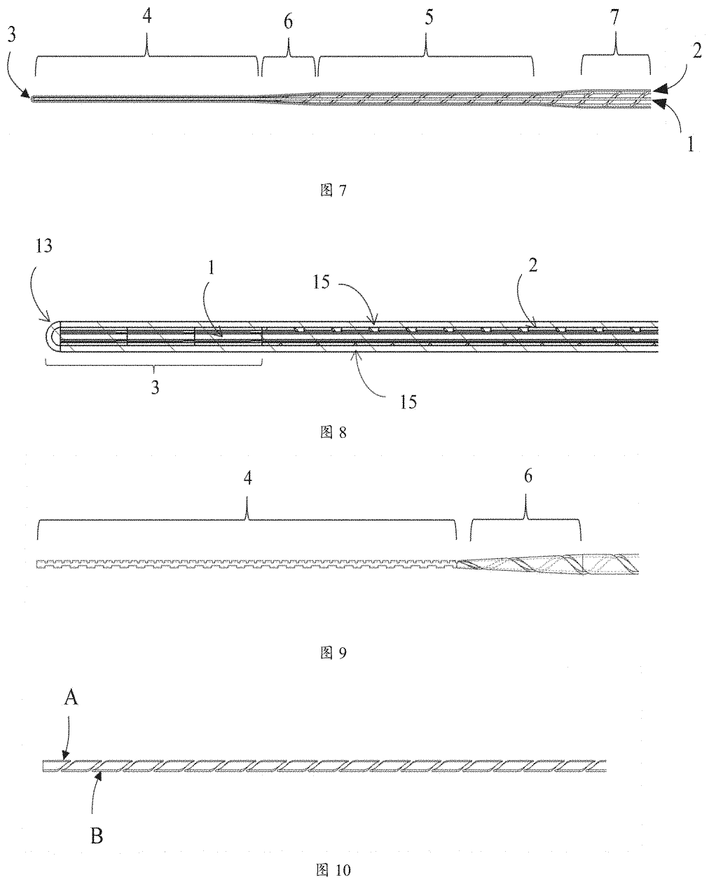

As shown in FIGS. 2 and 6, the functional section 3 of the optical fiber 1 may be connected to the shaping section 4 of the sleeve 2 via a spiral tube 11. The spiral tube 11 is preferably made of a metal material. A developing ring 12 may be provided between the spiral tube 11 and the optical fiber 1. The developing ring 12 may be fixed to the spiral tube 11 at the outside thereof by means of adhesives, and fixed to the optical fiber 1 at the inside thereof also by means of adhesives. The developing ring 12 is made of precious metals, such as gold, platinum, which can present clear images under the irradiation of X-rays, thereby assisting detection and treatment.

The optical functional guidewire may also be provided with a polymer coating 13. The polymer coating 13 may be a hydrophilic coating or a hydrophobic coating. The hydrophilic coating can capture water to form a “gel-like” surface on the surface of the guidewire, reducing a resistance during the passage of the guidewire. The hydrophobic coating can resist water molecules to form a “waxy” surface, reducing friction during the passage, and enhancing the tracking of the guidewire.

In this example, the sleeve 2 may be an equal-diameter sleeve 2 or a variable-diameter sleeve 2. When it is the equal-diameter sleeve 2, diameters of the shaping section 4 and the supporting section 5 of the sleeve 2 are equal. When it is the variable-diameter sleeve 2, the diameters of the shaping section 4 and the supporting section 5 of the sleeve 2 increase sequentially. In the practices, the sleeve 2 is preferably a variable-diameter sleeve 2. The diameter of the shaping section 4 is less than the diameter of the supporting section 5, and thus is more bendable compared to the supporting section 5, which prompts the moving along the bent blood vessel. The supporting section 5 has the larger diameter, and thus bring out sufficient rigidity, which can provide forward driving force for the shaping section 4.

In addition, the shaping section 4 and the supporting section 5 of the sleeve 2 may be equal-diameter sections or variable-diameter sections. In the case that shaping section 4 and/or the supporting section 5 is/are variable-diameter section, the diameter of each section gradually increases along the direction from the shaping section 4 to the supporting section 5. However, regardless of whether that shaping section 4 and the supporting section 5 are equal-diameter sections or variable-diameter section, the outer diameters thereof are different. The outer diameter of the shaping section 4 is always less than the outer diameter of the supporting section 5.

As shown in FIG. 7, in the optical functional guidewire provided in this example, the sleeve 2 further includes a transition section 6 and a pushing section 7. The transition section 6 is located between the shaping section 4 and the supporting section 5, and a diameter of the transition section 6 gradually increases in a direction from the shaping section 4 to the supporting section 5. An end of the pushing section 7 is connected to the supporting section 5, and other end of the pushing section 7 is connected to a driving mechanism, so as to provide forward driving force. The driving mechanism may be an operating handle that manually drives the optical functional guidewire to move, or a machine such as the attitude controller that electrically drives the optical functional guidewire to move, which is not limited in this disclosure.

The optical functional guidewire is further provided with an asymmetric structure capable of directional bending of the optical fiber guidewire to one side. The asymmetric structure is preferably an asymmetric tube wall structure of the sleeve 2, such as asymmetric slits 15 and asymmetric tube walls thickness or shape. The configuration of the asymmetric structure can make the optical fiber guidewire easier to bend to one side, improve the bending performance and operability of the optical functional guidewire. Therefore, it is easy to manipulate the optical functional guidewire into smaller blood vessels or branch blood vessels with larger opening angles for detection and treatment.

In this example, the optical functional guidewire preferably has a total length of 2m. The pushing section 7 of the sleeve 2 has a total outer diameter of preferably 0.8 mm, a length of 1 m. The sleeve 2 is preferably made of Medical 304 stainless steel. The supporting section 5 of the sleeve 2 can be formed by stretching the pushing section 7, has an outer diameter of preferably 0.4 mm, an inner diameter of preferably 0.3 mm, and a length of preferably 0.8 m. The transition section 6 and shaping section 4 of the sleeve 2 can also be formed by stretching the pushing section 7. The transition section 6 has a length of preferably 0.1 m. the shaping section 4 has an outer diameter of preferably 0.2 mm, an inner diameter of preferably 0.15 mm, and a length of preferably 0.1 m. In this example, the optical functional guidewire has the diameter of millimeters, so that it can safely enter the smaller blood vessel for detection or treatment, avoid the damage of the guidewire to walls of blood vessels, and has a wide range of applications.

In the practices, the optical fiber 1 of the optical functional guidewire can be connected to a multi-wavelength pulsed laser and a waveform collector through a fiber splitting coupler. The optical functional guidewire, at an end away from the functional section 3, can be connected to an attitude controller. The multi-wavelength pulse laser, the waveform collector and the attitude controller are all controlled by a control center. The control center sends control signals to the attitude controller, and the attitude controller controls the optical functional guidewire to enter into or exit the body cavity or move in the body cavity according to these control signals. The control center sends control signals to the multi-wavelength pulse laser, and the multi-wavelength pulse laser, based on these control signals emits pulsed laser light, and scatters the pulsed laser light into the cavity via the optical functional guidewire. The control center sends control signals to the waveform collector, and the waveform collector collects the delayed waveform of the scattered laser light based on these control signals, and then by calculation, obtains information about the distance between the wall of the body cavity and the optical functional guidewire including the relative position, whether there is a branch cavity in front of the fiber guidewire. The waveform collector feedbacks the above distance information to the control center and the attitude controller, so that the attitude and the next direction of movement of the guidewire can be controlled and adjusted, avoiding damage to the wall of the body cavity during the movement of the guidewire.

The optical functional guidewire provided in this disclosure includes at least one optical fiber 1 and a sleeve 2 surrounding the optical fiber 1. The optical fiber 1 includes a functional section 3 of emitting and collecting laser light. The functional section 3 is provided with at least one grating assembly 8. The grating assembly 8 has a function of emitting and collecting detection laser light, and can be used for determining the distance between the wall of the cavity and the optical fiber 1 by emitting and collecting lasers having different specific wavelengths and analyzing the time waveform obtained thereby, so as to guide the optical functional guidewire to change the shape and attitude over time, thereby realizing the intelligent-guidance and detection in the body cavity. The sleeve 2 includes the functional section 3, the guiding section and the supporting section 5 which are connected in sequence. In addition, in order to improve the bending performance and operability of the optical functional guidewire, the asymmetric structure is provided on the sleeve 2 itself or the surround of the sleeve 2 along the optical fiber 1, so that the optical functional guidewire can be easily manipulated and can readily enter the body cavity with a large opening angle, and precise detection and treatment can be carried out in the cavity through laser transmission, thereby improving the effect of minimally invasive interventional treatment.

It should be noted that the optical functional guidewire provided in this disclosure may also have diagnostic and therapeutic functions. For example, in the photodynamic therapy, after the above-mentioned guiding process, the guidewire is guided to the lesion site, and then the therapeutic red light can be emitted through the grating assembly 8 to excite singlet oxygen. The photosensitive drug emits fluorescence, and then the fluorescence spectrum can be collected and analyzed by the grating, which plays diagnostic effects. After completing the diagnosis process, the photodynamic laser for treatment is guided to excite the photosensitive drug, which has a therapeutic function with good therapeutic effect

### Example 2

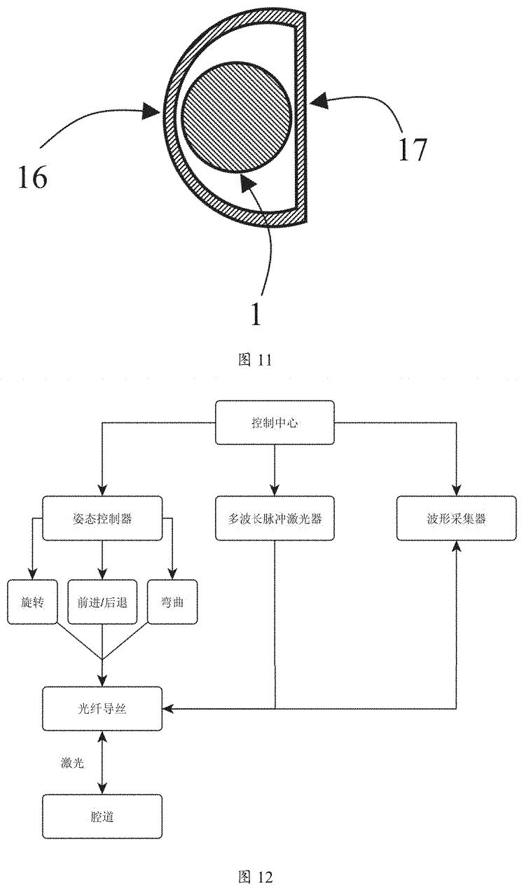

On the basis of example 1, the present example provides an optical functional guidewire having a cross-sectional structure of the shaping section 4 and functional section 3 shown in FIG. 8.

In this example, the asymmetric tube wall structure is the asymmetric slit 15 opened on the sleeve 2. The asymmetric slit 15 is a spiral slit. The asymmetric slit 15 has different widths on two sides of the sleeve 2. The asymmetrical slit 15 is preferably opened on the shaping section 4 of the sleeve 2. The slit has a smaller width on one side, and has a larger width on the other side. Thereby, the shaping section 4 can be bent toward the side having the larger slit when force is applied, and the flexibility of the optical fiber guidewire can be improved.

In the practices, the spiral slit of the sleeve can be formed by rotating cut by laser cutting process. The slit of the supporting section 5 has a width of preferably 0.5 mm, and a thread pitch of preferably 1 mm. The slit of shaping section 4 has a width of preferably 0.1 mm, and a thread pitch of preferably 0.5 mm.

In the optical functional guidewire provided in this example, the configuration of the spiral asymmetric slit 15 can further improve the bending performance and operability of the optical functional guidewire. Therefore, the optical functional guidewire can be easily manipulated and can readily enter the cavity with a large opening angle, realizing the self-guidance and flexible detection of the optical functional guidewire in the cavity, and improving the therapeutic effect of minimally invasive interventional therapy.

### Example 3

On the basis of example 1, the present example provides an optical functional guidewire having a side-sectional structure of the shaping section 4 and the supporting section 5 shown in FIG. 9.

In this example, the asymmetric tube wall structure is an asymmetric slit 15 opened on the sleeve 2, and the asymmetric slit 15 is a rectangular slit, i.e., the asymmetric slits 15 has different depths on two sides of the sleeve 2. The asymmetric slit 15 is preferably opened on the shaping section 4 of the sleeve 2. The asymmetric slit 15 of the shaping section 4 can make the optical fiber guidewire have asymmetric mechanical properties. It will be bent toward the side having the deeper depth when force is applied. Therefore, the optical functional guidewire can easily and quickly enter the body cavity with a larger opening angle. In addition, the rectangular slit is simple in the manufacture, is easy to be controlled during the use, and has high maneuverability and wide applications.

In the optical functional guidewire provided in this example, the configuration of the rectangular asymmetric slit 15 can further improve the bending performance and operability of the optical functional guidewire. Therefore, the optical functional guidewire can be easily manipulated and can readily enter the cavity with a large opening angle, realizing the self-guidance and flexible detection of the optical fiber guidewire in the cavity, and improving the therapeutic effect of minimally invasive interventional therapy.

### Example 4

On the basis of example 1, this example provides an optical functional guidewire having a side-sectional structure of the shaping section 4 shown in FIG. 10.

An asymmetric tube wall structure lies in the thickness of the sleeve, i.e., formed by a wall thickness on one side of the sleeve 2 being smaller than a wall thickness of other side of the sleeve 2. Specifically, taking the sleeve 2 being a cylindrical sleeve 2 as an example, if it is divided into two half-cylindrical sleeves 2 along the cross-sectional diameter, as shown in FIG. 10, A represents the tube wall having a thinner thickness which is preferably 0.1 mm-0.3 mm, while B represents the tube wall having a thicker thickness which is preferably 0.3 mm-0.5 mm.

In the optical functional guidewire provided in this example, the sleeve 2 has a thinner thickness on one side, and has a thicker thickness on the other side. When the guidewire is stressed, it will bend to the side of tube wall having the thinner thickness, so as to advance into the cavity with a larger opening angle.

In the optical functional guidewire provided in this example, the configuration of the asymmetric tube wall structure can further improve the bending performance and operability of the optical functional guidewire. Therefore, the optical functional guidewire can be easily manipulated and can easily enter the cavity with a large opening angle, realizing the self-guidance and flexible detection of the optical functional guidewire in the cavity, and improving the therapeutic effect of minimally invasive interventional therapy.

### Example 5

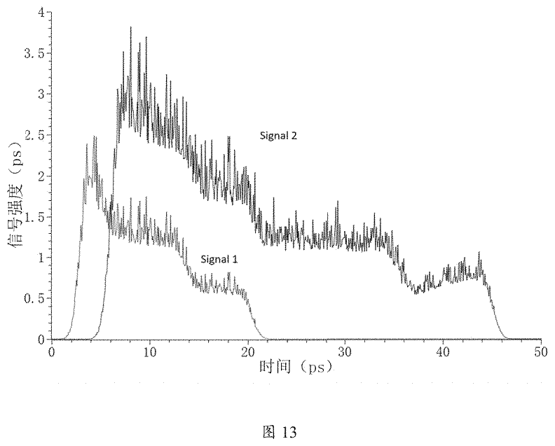

On the basis of example 1, the present example provides an optical functional guidewire having a cross sectional structure shown in FIG. 11.

The asymmetric tube wall structure lies in the shape of the sleeve, i.e., formed by a convex side 16 and a planar side 17 of the sleeve 2, or by a convex side 16 and a concave side of the sleeve 2. The convex side 16 has an arched structure.

Specifically, because the convex side 16 has the arched structure and its rigidity is relatively strong, when the optical functional guidewire is stressed, it will bend to the concave side or the planar side 17 opposite to the convex side 16, thereby making the optical functional guidewire advance into the curved cavity more smoothly.

In the optical functional guidewire provided in this example, the configuration of the asymmetric tubular structure can further improve the bending performance and operability of the optical functional guidewire. Therefore, the optical functional guidewire can be easily manipulated and can easily enter the cavity with a large opening angle, realizing the self-guidance and flexible detection of the optical functional guidewire in the cavity, and improving the therapeutic effect of minimally invasive interventional therapy.

### Example 6

The present example provides a detection system, including:

the optical functional guidewire as mentioned in any of examples 1-5;

a control center arranged for sending control signals to an attitude controller, a multi-wavelength pulsed laser, a waveform collector, and a treatment laser to control a start-up, operation or shutdown of the attitude controller, the multi-wavelength pulsed laser, the waveform collector, and the treatment laser;

the attitude controller arranged for receiving the signals sent by the control center and distance information, and driving the optical functional guidewire to entry or exit a body cavity or move in the body cavity;

the multi-wavelength pulsed laser arranged for receiving the signals sent by the control center, and sending out pulsed laser light which is transmitted to the optical functional guidewire, and is scattered into the body cavity through the grating assembly 8 of the optical functional guidewire;

the waveform collector arranged for receiving the signals sent by the control center, analyzing a delayed waveform of scattered laser in the body cavity through the grating assembly of the optical functional guidewire, to obtain position information about a wall of the cavity and the optical fiber guidewire, and feedback the position information to the control center.

The optical fiber 1 of the optical functional guidewire may be connected to the multi-wavelength pulsed laser and the waveform collector through a fiber splitting coupler. The optical functional guidewire, at an end close to the supporting section 5, can be connected to the attitude controller. The multi-wavelength pulsed laser, the waveform collector and the attitude controller are all controlled by a control center.

The control center sends control signals to the attitude controller, and the attitude controller controls the optical functional guidewire to enter into or exit the cavity or move in the cavity based on the received control signals. For example, a linear stepping motor may be used for driving the guidewire to move forward or backward; a stepper motor, steering gear or the like may be used for driving the rotation of the guidewire by the rotation; a linear stepping motor may be used for pulling the optical fiber, and driving the shaping section 4 to bend to the side with the larger slit.

The control center sends control signals to the multi-wavelength pulsed laser, and the multi-wavelength pulsed laser, based on these control signals emits pulsed laser light, and scatters the pulsed laser light into the cavity via the optical functional guidewire and grating assembly 8 thereof. The control center sends control signals to the waveform collector, and the waveform collector collects the delayed waveform of the scattered laser light by the grating assembly based on these control signals, and then by calculation, obtains information about the distance between the wall of the cavity and the optical fiber, including the relative position, whether there is a branch cavity in front of the fiber guidewire. When controlling the bending of the top portion, the optical functional guidewire can be tensioned by applying a pulling force through a tensioning mechanism.

The pulling force can be transmitted to the developing ring 12 of the shaping section 4 via the optical functional guidewire, and then transmitted to the sleeve having asymmetric structure via the developing ring 12, which causing the sleeve 2 to bend laterally.

Specifically, a specific example is provided for showing related calculation on the position in detail. For example, the multi-wavelength pulsed laser may emit picosecond pulses of 18 wavelengths (for example, 18 wavelengths increase sequentially in range of 1020 nm-1080 nm) with a single pulse width of 1 picosecond. The 18 pulses with 18 wavelengths enter the optical function guidewire after being combined by 18-1 beam combiners, and are respectively emitted through 18 gratings. The echo of the picosecond pulse scattered by the cavity wall is collected by the grating having a corresponding wavelength, and then returned to the waveform collector. The distance resolution corresponding to 1 picosecond pulse is 1E−12*1E8*1.33/2=66.5 μm. As shown in FIGS. 3-5 and FIG. 13, laser light emitted by grating a, scatters echoes by reflection from the cavity wall at different distances, which forms the waveform shown in FIG. 13 after being superimposed over time. The laser light emitted by grating b, scatters echoes by reflection from the cavity wall at different distances, which forms the waveform shown in FIG. 13 after being superimposed over time. Since the grating b is closer to the cavity wall, and there is a branch cavity at the grating a, the time period of Signal 2 obtained from the time delay superimposed by echoes is longer than the time period of Signal 1. Therefore, according to the time waveform analysis obtained by the control center, it can be determined that there is a branch cavity at grating group a.

The detection system provided by this example includes the optical functional guidewire, the control center, the attitude controller, the multi-wavelength pulsed laser, and the waveform collector. Among them, the control center can send control signals to other components to coordinate and control the cooperation among these components. The attitude controller can control the optical functional guidewire to enter or exit the body cavity or move in the body cavity, which improves the flexibility of the optical functional guidewire during use. Through the cooperation of the multi-wavelength pulsed laser, the waveform collector and the optical functional guidewire, the relative position of the optical functional guidewire and the wall of the body cavity can be determined by the delay of the laser light, and then the subsequent attitude and moving direction of the optical functional guidewire can be accurately determined. The detection system provided by this disclosure innovatively uses light to guide the travel of the guidewire, and has high detection efficiency and good detection effect.

### Example 7

The present example provides a detection method used in the detection system of example 6. The method includes the following step S1 to step S5.

At step S1, the control center receives control instructions, and sends control signals to the attitude controller and the multi-wavelength pulsed laser based on the control instructions.

At step S2, the attitude controller receives the control signals sent by the control center, and drives the optical functional guidewire into a body cavity based on the control signals.

At step S3, the pulse detector receives the control signals sent by the control center, emits pulsed laser light, and scatters the pulsed laser light into the body cavity via the optical functional guidewire and the grating assembly 8.

At step S4, the optical fiber guidewire receives a reflected pulsed laser light and sends the reflected pulsed laser light to the waveform collector. Based on the reflected pulsed laser light, the waveform collector determines a position of the optical functional guidewire in the body cavity.

At step S5, the attitude controller controls a subsequent movement of the optical functional guidewire based on the position of the optical functional guidewire in the body cavity, until the optical functional guidewire reaches target site and exits the body cavity after completing detection.

The detection method provided in this example realizes the intelligent and automatic guidance of the optical functional guidewire in the body cavity through the cooperation of the control center, the attitude controller, the pulsed detector, the optical functional guidewire and the waveform collector. This method is easy and convenient in operation, and greatly improves the detection efficiency and detection effect of the optical functional guidewire. In addition, through the cooperation of the control center, the optical functional guidewire and the treatment laser, the laser irradiation treatment can be performed to the lesion sites of the patient with high treatment efficiency and good effect, thereby improving flexibility in use and application range of the optical functional guidewire.

In the description, the expressions “equal”, “same” and the like are not strictly mathematical and/or geometrical limitations, and also include tolerances in manufacturing or use that can be understood by those skilled in the art.

Unless otherwise specified, the numerical range herein includes not only the entire range within its two endpoints, but also several sub-ranges contained therein.

Although preferred embodiments and examples of the present disclosure have been shown in details with reference to drawing, the present disclosure is not limited to the above embodiments and examples. Various modifications may be made without departing from the concept of the present disclosure, within the knowledge possessed by those skilled in the art.

1. An optical functional guidewire, comprising an optical fiber (1) and a sleeve (2) surrounding the optical fiber (1), wherein the optical fiber (1) comprises a functional section (3) capable of emitting and collecting laser light, and the functional section (3) is provided with at least one grating assembly (8); the sleeve (2) comprises a shaping section (4) capable of bending and a supporting section (5) capable of supporting an advancement of the functional section (3);
wherein the shaping section (4) is connected to the functional section (3) and is located at an end close to the functional section (3), the supporting section (5) is located at an end away from the functional section (3);
the optical functional guidewire is further provided with an asymmetric structure capable of directional bending of the optical functional guidewire.

2. The optical functional guidewire according to claim 1, wherein the functional section (3) is provided with a plurality of grating assemblies (8), and the grating assemblies are sleeved on the functional section (3) of the optical functional guidewire at intervals, and are arranged longitudinally along the optical fiber (1).

3. The optical functional guidewire according to claim 2, wherein the optical fiber (1) comprises a core layer (9) located at an axis, and a cladding layer (10) surrounding the core layer (9); and
the grating assemblies (8) are sleeved outside the cladding layer (10), and each of grating assemblies (8) is in a shape of hollow prism.

4. The optical functional guidewire according to claim 3, wherein the grating assembly (8) comprises a plurality of gratings with different periods, and each grating constitutes a side surface of the grating assembly (8).

5. The optical functional guidewire according to claim 1, wherein a diameter of the supporting section (5) is larger than a diameter of the shaping section (4).

6. The optical functional guidewire according to claim 1, wherein the functional section (3) of the optical fiber (1) is connected to the shaping section (4) of the sleeve (2) via a spiral tube (11), and a developing ring (12) is provided between the spiral tube (11) and the optical fiber (1).

7. The optical functional guidewire according to claim 1, wherein the asymmetric structure is an asymmetric tube wall structure of the sleeve (2).

8. The optical functional guidewire according to claim 1, wherein the functional section (3), at an end away from the shaping section (4), is provided with a hemispherical optical component (14) capable of blocking laser scattering;
a polymer coating (13) is provided outside the optical functional guidewire, and the polymer coating (13) is a hydrophilic coating or a hydrophobic coating.

9. A detection system, comprising:
the optical functional guidewire according to claim 1;
a control center arranged for sending control signals to an attitude controller, a multi-wavelength pulsed laser, a waveform collector, and a treatment laser to control a start-up, operation or shutdown of the attitude controller, the multi-wavelength pulsed laser, the waveform collector, and the treatment laser;
the attitude controller arranged for receiving the signals sent by the control center and distance information, and driving the optical functional guidewire to entry or exit a body cavity or move in the body cavity;
the multi-wavelength pulsed laser arranged for receiving the signals sent by the control center, and sending out pulsed laser light which is transmitted to the optical functional guidewire, and is scattered into the body cavity through the grating assembly (8) of the optical functional guidewire;
the waveform collector arranged for receiving the signals sent by the control center, analyzing a delayed waveform of scattered laser in the body cavity through the grating assembly (8) of the optical functional guidewire, to obtain position information about a wall of the body cavity and the optical functional guidewire, and feedback the position information to the control center.

10. A detection method used in the detection system according to claim 9, wherein the method comprising:
by the control center, receiving control instructions, and sending control signals to the attitude controller and the multi-wavelength pulsed laser based on the control instructions;
by the attitude controller, receiving the control signals sent by the control center, and driving the optical functional guidewire into a body cavity based on the control signals;
by the pulse detector, receiving the control signals sent by the control center, emitting pulsed laser light; and via the optical functional guidewire and the grating assembly (8), scattering the pulsed laser light into the body cavity;
by the optical functional guidewire, receiving a reflected pulsed laser light and sending the reflected pulsed laser light to the waveform collector; and by the waveform collector, based on the reflected pulsed laser light, determining a position of the optical functional guidewire in the body cavity; and
by the attitude controller, controlling a subsequent movement of the optical functional guidewire based on the position of the optical functional guidewire in the body cavity, until the optical functional guidewire reaches target site and exits the body cavity after completing a detection.

11. The optical functional guidewire according to claim 5, wherein the sleeve (2) further includes a transition section (6) and a pushing section (7);
the transition section (6) is located between the shaping section (4) and the supporting section (5), and a diameter of the transition section (6) gradually increases in a direction from the shaping section (4) to the supporting section (5); and
an end of the pushing section (7) is connected to the supporting section (5), and other end of the pushing section (7) is connected to a driving mechanism.

12. The optical functional guidewire according to claim 8, wherein the asymmetric tube wall structure is an asymmetric slit (15) opened on the shaping section (4) of the sleeve (2), an asymmetric tube wall thickness of the sleeve (2), or a shape of the sleeve (2).
